(19) United States
(12) Patent Application Publication
(10) Pub. No.: US 2022/0339090 A1
(43) Pub. Date: Oct. 27, 2022

(54) RETINAL-CONTAINING MULTILAMELLAR VESICLE AND COSMETIC COMPOSITION COMPRISING SAME

(71) Applicant: DAREUN COSMETICS CO., LTD., Seoul (KR)

(72) Inventors: Dong Won LEE, Seoul (KR); Hong Geun JI, Gyeonggi-do (KR)

(21) Appl. No.: 17/762,255

(22) PCT Filed: Sep. 23, 2020
(86) PCT No.: PCT/KR2020/012829

§ 371 Date: Mar. 21, 2022

(30) Foreign Application Priority Data
Sep. 30, 2019 (KR) 10-2019-0120447

(51) Int. Cl. A61K 8/67; A61K 8/14; A61K 8/63; A61K 8/44; A61K 8/37; A61K 8/55; A61K 8/42; A61K 8/31; A61K 8/34; A61K 8/92; A61Q 19/00

(52) U.S. Cl. CPC A61K 8/671; A61K 8/14; A61K 8/63; A61K 8/44; A61K 8/375; A61K 8/553; A61K 8/42; A61K 8/31; A61K 8/345; A61K 8/922; A61Q 19/00

(57) ABSTRACT

The present invention relates to a retinal-containing multilamellar vesicle and, more specifically, to a retinal-containing multilamellar vesicle comprising 0.01-5% by weight of retinal, 0.1-10% by weight of rapeseed sterol, 0.1-10% by weight of phytosteryl/behenyl/octyldodecyl lauroyl glutamate, 0.1-8% by weight of polyglyceryl-10 oleate, 0.1-10% by weight of cholesterol, 0.01-5% by weight of hydrogenated lecithin, 0.01-5% by weight of ceramide, 1-15% by weight of squalane, 0.1-10% by weight of vegetable butter, 3-30% by weight of oily liquid, 1-20% by weight of glycerin, and 30-70% by weight of water, and to a cosmetic composition comprising same.

### TECHNICAL FIELD

The present invention relates to a multilamellar vesicle containing retinal. More specifically, the present invention relates to a multilamellar vesicle containing retinal which comprises 0.01 to 5% by weight of retinal, 0.1 to 10% by weight of rapeseed sterol, 0.1 to 10% by weight of phytosteryl/behenyl/octyldodecyl lauroyl glutamate, 0.1 to 8% by weight of polyglyceryl-10 oleate, 0.1 to 10% by weight of cholesterol, 0.01 to 5% by weight of hydrogenated lecithin, 0.01 to 5% by weight of ceramide, 1 to 15% by weight of squalane, 0.1 to 10% by weight of vegetable butter, 3 to 30% by weight of oily liquid, 1 to 20% by weight of glycerin and 30 to 70% by weight of water.

In addition, the present invention relates to a cosmetic composition comprising the multilamellar vesicle containing retinal.

### BACKGROUND ART

Cosmetic consumers are demanding high efficacy and effect as well as safety and avoidance of skin irritation. Due to this, the term “cosmeceutical”—which is a combination of cosmetics and pharmaceutical—has emerged in accordance with consumers' desire for high-functional cosmetics. With the development of cosmeceuticals, the number of products that combine technologies in other fields such as medical technology to cosmetics is increasing. Due to the development of such functional materials, various functionalization methods that can give higher stability to cosmetic raw materials are being extensively studied. Specifically, it is well known that light, heat and oxygen in the air seriously reduce the biological activity of functional raw materials. Therefore, there is a need for a new functionalization technology for stabilizing various cosmetic raw materials.

Vitamin A is involved in visual function and acts as a growth factor. Vitamin A acts in the retina of the eye in the form of a metabolite, the light-absorbing pigment molecule retinal, which is absolutely necessary for twilight vision (the ability to see objects in the dark) and color perception. Vitamin A also functions as an important epidermal growth factor like a hormone in epithelial cells in the form of retinoic acid, which is an irreversibly oxidized form of retinol. Most of the vitamin A in animal foods exists in the form of esters such as retinyl palmitate. After ingestion with food, vitamin A is converted into retinol, a type of alcohol, in the small intestine. The human body stores vitamin A in the form of retinol and converts it into retinal (retinaldehyde)—which is an aldehyde that acts in the visual system—whenever needed. Both retinol and retinal are precursors of tretinoin (all-trans retinoic acid) and are synthesized through retinoid metabolism (FIG. 1). The place marked as “rate limiting” in FIG. 1 is a stage in which retinol is transferred to retinal, and this stage is the most difficult stage in retinoid metabolism. That is, no matter how high concentration of retinol is applied, this step cannot be passed, and thus the concentration of tretinoin does not increase in proportion to the amount of retinol. However, because retinal has passed this stage, it is likely to be converted to tretinoin easily. Retinal helps to remove comedo (blackheads, whiteheads) by controlling keratinization of the skin, helps to improve skin wrinkles and skin texture, and can improve skin barrier function and retention of moisture.

However, since retinal is unstable to heat, light and moisture, and not easily absorbed into the skin, there is a need for improving these matters. As an example for stabilizing retinal, Korean Patent Application Publication No. 10-2018-0003356 discloses a cosmetic composition containing a retinal liposome which comprises a liposome dispersed in an aqueous component, wherein the liposome comprises a phospholipid layer comprising a lecithin compound and a surfactant; and a carried body entrapped in the phospholipid layer, and comprising retinal, ceramide and pentaerythrityl tetra-di-t-butyl hydroxyhydrocinnamate in a weight ratio of 1:5 to 20:0.1-3; and the surfactant comprises at least one of a nonionic surfactant and an anionic surfactant.

### SUMMARY

### Technical Problem

Therefore, the technical problem of the present invention is the provision of a delivery system containing retinal which can efficiently deliver retinal into the skin in a stable manner.

In addition, another technical problem of the present invention is the provision of a cosmetic composition comprising the delivery system containing retinal.

### Solution To Problem

To solve the above technical problem, the present invention provides a multilamellar vesicle containing retinal which comprises 0.01 to 5% by weight of retinal, 0.1 to 10% by weight of rapeseed sterol, 0.1 to 10% by weight of phytosteryl/behenyl/octyldodecyl lauroyl glutamate, 0.1 to 8% by weight of polyglyceryl-10 oleate, 0.1 to 10% by weight of cholesterol, 0.01 to 5% by weight of hydrogenated lecithin, 0.01 to 5% by weight of ceramide, 1 to 15% by weight of squalane, 0.1 to 10% by weight of vegetable butter, 3 to 30% by weight of oily liquid, 1 to 20% by weight of glycerin and 30 to 70% by weight of water.

In addition, the present invention provides a cosmetic composition comprising the multilamellar vesicle containing retinal.

The present invention is described in detail hereinafter.

According to one aspect of the present invention, there is provided a multilamellar vesicle containing retinal which comprises 0.01 to 5% by weight of retinal, 0.1 to 10% by weight of rapeseed sterol, 0.1 to 10% by weight of phytosteryl/behenyl/octyldodecyl lauroyl glutamate, 0.1 to 8% by weight of polyglyceryl-10 oleate, 0.1 to 10% by weight of cholesterol, 0.01 to 5% by weight of hydrogenated lecithin, 0.01 to 5% by weight of ceramide, 1 to 15% by weight of squalane, 0.1 to 10% by weight of vegetable butter, 3 to 30% by weight of oily liquid, 1 to 20% by weight of glycerin and 30 to 70% by weight of water.

In the present invention, retinal can be efficiently delivered into the skin in a stable manner by preparing multilamellar vesicles (MLVs) using rapeseed sterol, phytosteryl/behenyl/octyldodecyl lauroyl glutamate, polyglyceryl-10 oleate, cholesterol, hydrogenated lecithin, ceramide, squalane, vegetable butter, oily liquid, glycerin and water.

The multilamellar vesicle containing retinal according to the present invention comprises retinal (retinaldehyde) in an amount of 0.01 to 5% by weight, preferably 0.02 to 4% by weight and more preferably 0.5 to 3% by weight. In the multilamellar vesicle containing retinal of the present invention, if the amount of retinal is less than 0.01% by weight, the efficacy according to retinal may be weak, and if the amount of retinal is more than 5% by weight, there may be a problem in the formation of multilamellar vesicles.

The multilamellar vesicle containing retinal according to the present invention comprises rapeseed sterol in an amount of 0.1 to 10% by weight, preferably 0.5 to 8% by weight and more preferably 1 to 7% by weight. Rapeseed sterol is phytosterol, and commercially available—for example, by BASF Corporation under the trade name “Generol® R.” In the present invention, the rapeseed sterol imparts stability to the bilayer membrane of multilamellar vesicles. In the present invention, if the amount of rapeseed sterol is less than 0.1% by weight, the effect of imparting stability by rapeseed sterol may be weak, and if the amount of rapeseed sterol is more than 10% by weight, there may be a problem in the formation of multilamellar vesicles.

The multilamellar vesicle containing retinal according to the present invention comprises phytosteryl/behenyl/octyldodecyl lauroyl glutamate in an amount of 0.1 to 10% by weight, preferably 0.5 to 8% by weight and more preferably 1 to 7% by weight. Phytosteryl/behenyl/octyldodecyl lauroyl glutamate is a mixed ester of phytosterol, behenyl alcohol, octyldodecanol, and lauroyl glutamic add, and in the present invention, it can increase the stability of multilamellar vesicles. In the present invention, if the amount of phytosteryl/behenyl/octyldodecyl lauroyl glutamate is less than 0.1% by weight or more than 10% by weight, there may be a problem in the formation of multilamellar vesicles.

The multilamellar vesicle containing retinal according to the present invention comprises polyglyceryl-10 oleate in an amount of 0.1 to 8% by weight, preferably 0.2 to 7% by weight and more preferably 0.5 to 5% by weight. Polyglyceryl-10 oleate is an ester of polyglycerol-10 and oleic add. In the present invention, polyglyceryl-10 oleate forms the lipid bilayer of multilamellar vesicles together with hydrogenated lecithin. In the present invention, if the amount of polyglyceryl-10 oleate is less than 0.1% by weight or more than 8% by weight, there may be a problem in the formation of multilamellar vesicles.

The multilamellar vesicle containing retinal according to the present invention comprises cholesterol in an amount of 0.1 to 10% by weight, preferably 0.5 to 8% by weight and more preferably 1 to 7% by weight. In the present invention, cholesterol can help to form more stable multilamellar vesicles by imparting stability to the bilayer membrane of multilamellar vesicles. In the present invention, if the amount of cholesterol is less than 0.1% by weight, the effect of imparting stability by cholesterol may be weak, and if the amount of cholesterol is more than 10% by weight, there may be a problem in the formation of multilamellar vesicles.

The multilamellar vesicle containing retinal according to the present invention comprises hydrogenated lecithin in an amount of 0.01 to 5% by weight, preferably 0.05 to 4% by weight and more preferably 0.1 to 3% by weight. In the present invention, lecithin refers to a mixture of various phospholipids, and the composition of phospholipids may vary according to origin. Hydrogenated lecithin can be obtained by adding hydrogen to lecithin. In the present invention, if the amount of hydrogenated lecithin is less than 0.05% by weight or more than 4% by weight, there may be a problem in the formation of multilamellar vesicles.

The multilamellar vesicle containing retinal according to the present invention comprises ceramide in an amount of 0.01 to 5% by weight, preferably 0.05 to 4% by weight and more preferably 0.1 to 3% by weight. Ceramide is a lipid molecule composed of sphingosine and a fatty acid, and can impart flexibility to multilamellar vesicles and provide a moisturizing effect to the skin at the same time. In the present invention, if the amount of ceramide is less than 0.01% by weight, the effect provided by ceramide may be weak, and if the amount of ceramide is more than 5% by weight, there may be a problem in the formation of multilamellar vesicles.

The multilamellar vesicle containing retinal according to the present invention comprises squalane in an amount of 1 to 15% by weight, preferably 2 to 12% by weight and more preferably 3 to 10% by weight. Squalane can be obtained by hydrogenation of squalene, and can impart flexibility to multilamellar vesicles and provide a moisturizing effect to the skin at the same time. In the present invention, if the amount of squalane is less than 1% by weight, the effect provided by squalane may be weak, and if the amount of squalane is more than 15% by weight, there may be a problem in the formation of multilamellar vesicles.

The multilamellar vesicle containing retinal according to the present invention comprises vegetable butter in an amount of 0.1 to 10% by weight, preferably 0.5 to 8% by weight and more preferably 1 to 7% by weight.

In one embodiment according to the present invention, the example of vegetable butter includes, but is not limited to, shea butter, cocoa butter, olive butter, macadamia nut butter, coconut butter, almond butter or a mixture thereof.

In the present invention, if the amount of vegetable butter is less than 0.1% by weight or more than 10% by weight, there may be a problem in the formation of multilamellarvesicles.

The multilamellar vesicle containing retinal according to the present invention comprises oily liquid in an amount of 3 to 30% by weight, preferably 4 to 25% by weight and more preferably 5 to 20% by weight.

In one embodiment according to the present invention, the example of oily liquid includes, but is not limited to, safflower oil, sunflower seed oil, jojoba oil, olive oil, tea tree oil, canola oil, castor oil, palm oil, cetyl ethylhexanoate, isocetyl ethylhexanoate, cetyl isononanoate, cetearyl isononanoate, cetearyl nonanoate, ethylhexyl isononanoate, ethylhexyl isostearate or a mixture thereof.

In the present invention, if the amount of oily liquid is less than 3% by weight or more than 30% by weight, there may be a problem in the formation of multilamellar vesicles.

The multilamellar vesicle containing retinal according to the present invention comprises glycerin and water as solvents. The multilamellar vesicle containing retinal according to the present invention comprises glycerin in an amount of 1 to 20% by weight, preferably 2 to 18% by weight and more preferably 3 to 15% by weight. The multilamellar vesicle containing retinal according to the present invention comprises water in an amount of 30 to 70% by weight, preferably 35 to 68% by weight and more preferably 40 to 65% by weight. In the present invention, if the amount of glycerin and water is outside the above range, there may be a problem in the formation of multilamellarvesicles.

The multilamellar vesicle containing retinal according to the present invention consists essentially of the above ingredients of retinal, rapeseed sterol, phytosteryl/behenyl/octyldodecyl lauroyl glutamate, polyglyceryl-10 oleate, cholesterol, hydrogenated lecithin, ceramide, squalane, vegetable butter, oily liquid, glycerin and water, but may further comprise ingredients such as a stabilizer and an antioxidant within a scope that does not affect the essential properties of the multilamellar vesicles, if necessary. The average particle diameter of the multilamellar vesicle liposome according to the present invention is preferably 50 to 400 nm, and more preferably 100 to 350 nm.

According to another aspect of the present invention, there is provided a cosmetic composition comprising the multilamellar vesicle containing retinal.

In the present invention, the cosmetic composition may be formulated to toner, lotion, cream, essence and the like, but is not limited thereto. The cosmetic composition comprises preferably 1 to 60% by weight, and more preferably 3 to 55% by weight of the multilamellar vesicle containing retinal according to the present invention. In the present invention, if the cosmetic composition comprises the multilamellar vesicle containing retinal in an amount of less than 1% by weight, the effect according to retinal may be weak, and if the amount of the multilamellar vesicle containing retinal is greater than 60% by weight, it may be economically undesirable since increasing the effect according to retinal commensurately with the added amount would not be expected.

### Effects Of Invention

According to the present invention, retinal can be efficiently delivered into the skin in a very stable form through multilamellar vesicles having a structure similar to the skin, thereby increasing bioavailability and improving skin elasticity and skin wrinkles by retinal.

### BRIEF DESCRIPTION OF THE DRAWINGS

FIG. 1 is a schematic diagram showing retinoid metabolism.

FIG. 2 is a result of measuring the particle diameter size of multilamellar vesicles containing retinal by the use of Photal, ELS-Z.

FIG. 3 is a result of measuring zeta potential by the use of Photal, ELS-Z to measure the stability of multilamellar vesicles containing retinal.

FIG. 4 is a cryo-electron microscopy photograph of multilamellar vesicles containing retinal.

FIGS. 5A, 5B and 5C are graphs representing changes in retinal content measured in the cream of Comparative Example 2 (Cream A) and the cream of Example 4 (Cream B).

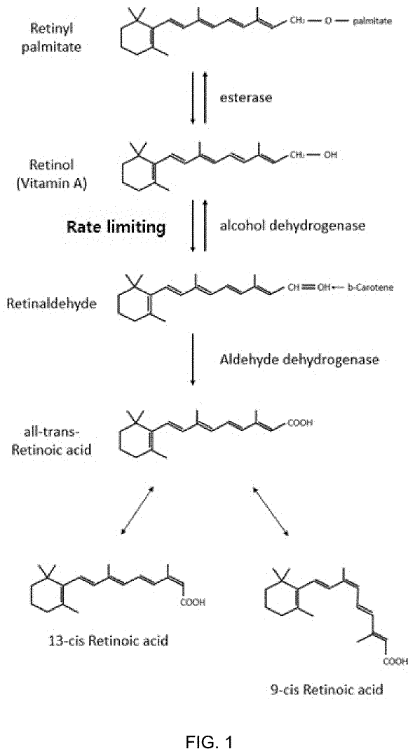

### DETAILED DESCRIPTION

Hereinafter, the present invention is explained in more detail with the following examples. However, it must be understood that the protection scope of the present invention is not limited to the examples.

### Example 1: Preparation of Multilamellar Vesicle Containing Retinal

According to the constitutional compositions of Table 1, the ingredients were introduced into a vessel and dissolved at 80° C. The resulting ingredients were mixed by the use of a homo-mixer for 5 minutes and passed three (3) times through a high-pressure homogenizer at 1,000 bar, followed by cooling and deaeration to obtain multilamellar vesicles in which retinal is stabilized.

TABLE 1

| Ingredient | Content (% by weight) |
|---|---|
| Retinal | 0.1 |
| Rapeseed sterol | 3 |
| Phytosteryl/behenyl/octyldodecyl lauroyl glutamate | 3 |
| Polyglyceryl-10 oleate | 2 |
| Cholesterol | 3 |
| Hydrogenated lecithin | 0.5 |
| Ceramide | 0.2 |
| Squalane | 5 |
| Shea butter | 3 |
| Safflower oil | 5 |
| Cetyl ethylhexanoate | 10 |
| Glycerin | 7 |
| Distilled water | 58.2 |
| Total amount | 100 |

### Comparative Example: Preparation of General Liposome containing Retinal

According to the constitutional composition of Table 2, the ingredients were introduced into a vessel and dissolved at 80° C. The resulting ingredients were mixed by the use of a homo-mixer for 5 minutes and passed three (3) times through a high-pressure homogenizer at 1,000 bar, followed by cooling and deaeration to obtain liposome composition.

TABLE 2

| Ingredient | Content (% by weight) |
|---|---|
| Retinal | 0.1 |
| Cholesterol | 3 |
| Hydrogenated lecithin | 0.5 |
| Ceramide | 0.2 |
| Squalane | 5 |
| Shea butter | 3 |
| Safflower oil | 5 |
| Cetyl ethylhexanoate | 10 |
| Glycerin | 7 |
| Distilled water | 66.2 |
| Total amount | 100 |

### Example 2: Preparation of Toner by Using Multilamellar Vesicle Containing Retinal

A toner was prepared by the use of multilamellar vesicle containing retinal according to the constitutional composition of Table 3.

TABLE 3

| Ingredient | Content (% by weight) |
|---|---|
| Multilamellar vesicle of Example 1 | 5 |
| 1,3-Butylene glycol | 7 |
| PEG-1500 | 5 |
| Hyaluronic acid | 1 |
| Natto extract | 5 |
| Distilled water | 77 |
| Total amount | 100 |

### Example 3: Preparation of Lotion by Using Multilamellar Vesicle Containing Retinal

A lotion was prepared by the use of multilamellar vesicle containing retinal according to the constitutional composition of Table 4.

TABLE 4

| Ingredient | Content (% by weight) |
|---|---|
| Multilamellar vesicle of Example 1 | 15 |
| Polysorbate 20 | 5 |
| Cetearyl alcohol | 0.5 |
| Olive oil | 3 |
| Squalane | 4 |
| Silicone oil | 2 |
| Butylene glycol | 5 |
| Carbopol | 0.2 |
| Distilled water | 65.3 |
| Total amount | 100 |

### Example 4: Preparation of Cream by Using Multilamellar Vesicle Containing Retinal

A cream was prepared by the use of multilamellar vesicle containing retinal according to the constitutional composition of Table 5.

TABLE 5

| Ingredient | Content (% by weight) |
|---|---|
| Multilamellar vesicle of Example 1 | 50 |
| Polyglyceryl-3 Stearate/Citrate | 4 |
| Behenyl alcohol | 1.5 |
| Safflower oil | 5 |
| Sunflower seed oil | 3 |
| Dimethicone oil | 2 |
| Propylene glycol | 8 |
| Carbopol | 0.36 |
| Distilled water | 26.14 |
| Total amount | 100 |

### Comparative Example 2: Preparation of Cream Containing Retinal

A cream containing retinal was prepared according to the constitutional composition of Table 6.

TABLE 6

| Ingredient | Content (% by weight) |
|---|---|
| Retinal undiluted solution | 0.05 |
| Polyglyceryl-3 stearate/citrate | 4 |
| Behenyl alcohol | 1.5 |
| Safflower oil | 5 |
| Sunflower seed oil | 3 |
| Dimethicone oil | 2 |
| Propylene glycol | 8 |
| Carbopol | 0.36 |
| Distilled water | 76.09 |
| Total amount | 100 |

### Example 5: Preparation of Essence by Using Multilamellar Vesicle Containing Retinal

An essence was prepared by the use of multilamellar vesicle containing retinal according to the constitutional composition of Table 7.

TABLE 7

| Ingredient | Content (% by weight) |
|---|---|
| Multilamellar vesicle of Example 1 | 20 |
| Carbopol | 0.3 |
| Hyaluronic acid | 3 |
| Allantoin | 0.1 |
| PEG-1500 | 3 |
| Calcium hydroxide | 0.03 |
| Sodium EDTA | 0.05 |
| Propylene glycol | 6 |
| Polyglyceryl-3 stearate/citrate | 5 |
| Distilled water | 62.52 |
| Total amount | 100 |

### Experimental Example 1: Measurement of Particle Size Distribution

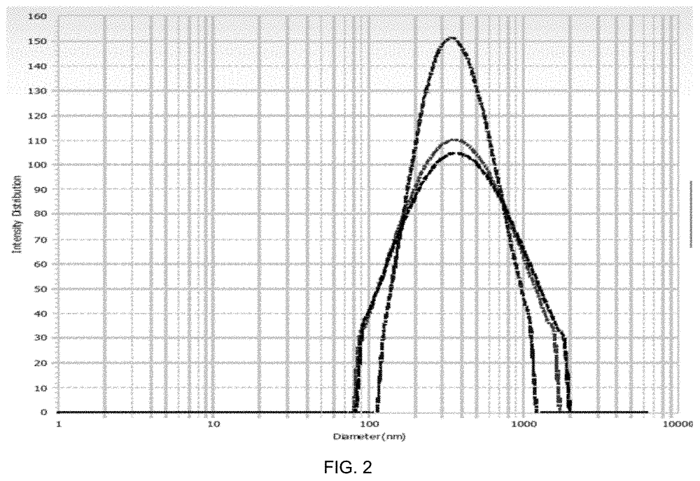

The particle size distribution of the multilamellar vesicles containing retinal prepared in Example 1 was measured by the use of Photal, ELS-Z, and the result is represented in FIG. 2. From the result of the measurement, it can be known that the average particle size of the multilamellar vesicles containing retinal is 308.1 nm.

### Experimental Example 2: Measurement of Stability of Multilamellar Vesicle Containing Retinal

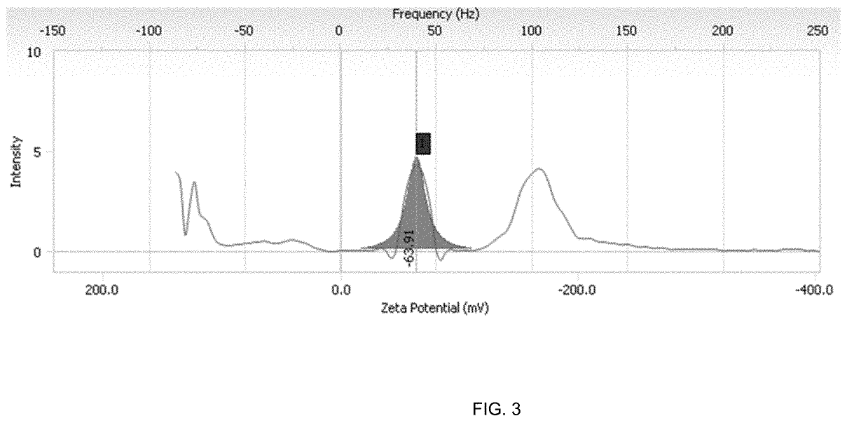

To measure the stability of the multilamellar vesicles containing retinal prepared in Example 1, zeta potential was measured by the use of Photal, ELS-Z, and the result is represented in FIG. 3. From the result of the measurement, it can be known that the potential is −63.91 mV, and the multilamellar vesicles are stable.

### Experimental Example 3: Electron Microscopy

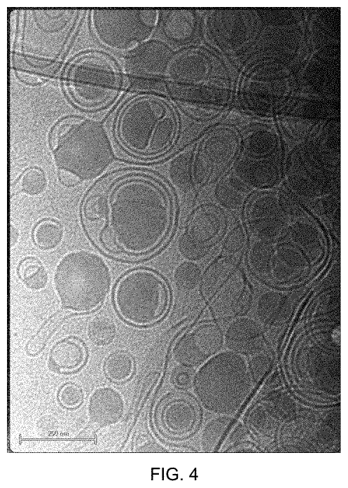

Photographs of the multilamellar vesicles containing retinal prepared in Example 1 were taken. Due to very fine particle size, it was impossible to take photographs by a general optical microscope. Therefore, cryo-electron microscopy photographs were taken (FIG. 4). From FIG. 4, it can be known that the multilamellar vesicles are well formed.

### Experimental Example 4: Test for Promoting Transdermal Absorption of Multilamellar Vesicle Containing Retinal

The artificial skin, Neoderm (Tego Science, Korea) was mounted to a Franz-type diffusion cell (Lab Fine Instruments, Korea). 50 mM phosphate buffer (pH 7.4, 0.1M NaCl) was added to a receptor cell (5 mL) of the Franz-type diffusion cell. A diffusion cell was then mixed and diffused at 600 rpm, 32° C., and 50 μL of the multilamellar vesicles of Example 1 and the liposome of Comparative Example 1, respectively, were added to donor cells. Absorption and diffusion were carried out according to the predetermined time, and the area of the skin where the absorption and diffusion were carried out was 0.64 cm². After finishing the absorption and diffusion of the active ingredient, the residues—which were not absorbed and remained on the skin—were cleaned with dried Kimwipes™ or 10 ml of ethanol. The skin in which the active ingredient was absorbed and diffused was homogenized by the use of a tip-type homogenizer, and retinal absorbed into the skin was then extracted with 4 ml of dichloromethane. The extract was then filtrated with a 0.45 μm nylon membrane filter. The content of retinal was measured by high-performance liquid chromatography with the following conditions, and the results are represented in Table 8.

TABLE 8

| | Transdermal absorption | Rate of |
|---|---|---|
| | (μg) | increase |
| Multilamellar vesicle of Example 1 | 0.3822 | 2-fold |
| Liposome of Comparative Example 1 | 0.2111 | — |

A) Column: C18 (4.6 × 200 mm, 5 μm)
B) Mobile phase: methanol: hexane = 2:1
C) Flow rate: 0.8 mL/min
D) Detector: UV 280 nm

As can be seen from Table 8, it can be known that the multilamellar vesicles of the present invention can deliver retinal into the skin very efficiently.

### Experimental Example 5: Analysis of Content Change

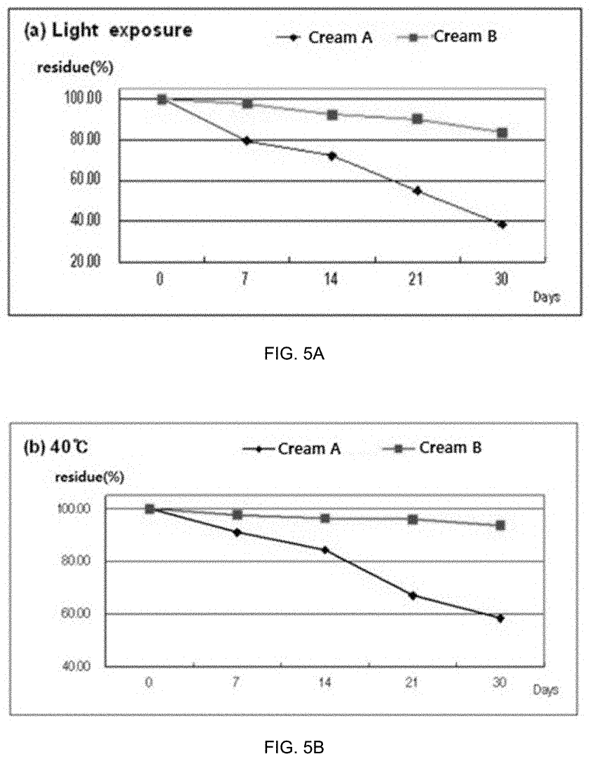

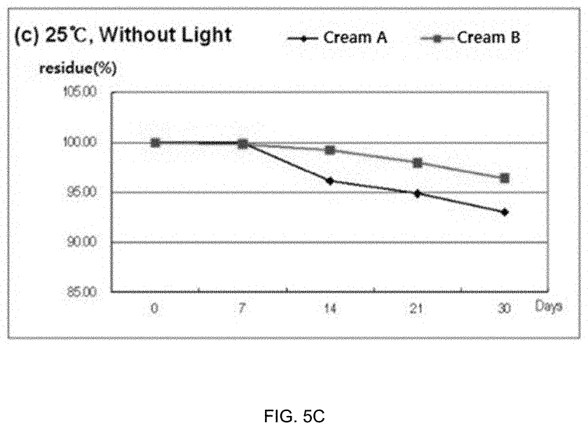

In order to evaluate the stability of retinal to light and temperature, the changes in the content of retinal in the cream of Comparative Example 2 (Cream A) and the cream of Example 4 (Cream B) were analyzed, and the results are represented in FIG. 5. Analysis conditions are the same as in Table 8 above, and HPLC (Model 510 Waters, USA) was used. As can be seen from FIG. 5, it can be known that the multilamellar vesicles of the present invention effectively stabilize retinal.

1. A multilamellar vesicle containing retinal which comprises 0.01 to 5% by weight of retinal, 0.1 to 10% by weight of rapeseed sterol, 0.1 to 10% by weight of phytosteryl/behenyl/octyldodecyl lauroyl glutamate, 0.1 to 8% by weight of polyglyceryl-10 oleate, 0.1 to 10% by weight of cholesterol, 0.01 to 5% by weight of hydrogenated lecithin, 0.01 to 5% by weight of ceramide, 1 to 15% by weight of squalane, 0.1 to 10% by weight of vegetable butter, 3 to 30% by weight of oily liquid, 1 to 20% by weight of glycerin and 30 to 70% by weight of water.

2. The multilamellar vesicle containing retinal according to claim 1, which comprises 0.02 to 4% by weight of retinal, 0.5 to 8% by weight of rapeseed sterol, 0.5 to 8% by weight of phytosteryl/behenyl/octyldodecyl lauroyl glutamate, 0.2 to 7% by weight of polyglyceryl-10 oleate, 0.5 to 8% by weight of cholesterol, 0.05 to 4% by weight of hydrogenated lecithin, 0.05 to 4% by weight of ceramide, 2 to 12% by weight of squalane, 0.5 to 8% by weight of vegetable butter, 4 to 25% by weight of oily liquid, 2 to 18% by weight of glycerin and 35 to 68% by weight of water.

3. The multilamellar vesicle containing retinal according to claim 1, which comprises 0.05 to 3% by weight of retinal, 1 to 7% by weight of rapeseed sterol, 1 to 7% by weight of phytosteryl/behenyl/octyldodecyl lauroyl glutamate, 0.5 to 5% by weight of polyglyceryl-10 oleate, 1 to 7% by weight of cholesterol, 0.1 to 3% by weight of hydrogenated lecithin, 0.1 to 3% by weight of ceramide, 3 to 10% by weight of squalane, 1 to 7% by weight of vegetable butter, 5 to 20% by weight of oily liquid, 3 to 15% by weight of glycerin and 40 to 65% by weight of water.

4. The multilamellar vesicle containing retinal according to claim 1, wherein the vegetable butter is selected from the group consisting of shea butter, cocoa butter, olive butter, macadamia nut butter, coconut butter, almond butter and a mixture thereof.

5. The multilamellar vesicle containing retinal according to claim 1, wherein the oily liquid is selected from the group consisting of safflower oil, sunflower seed oil, jojoba oil, olive oil, tea tree oil, canola oil, castor oil, palm oil, cetyl ethylhexanoate, isocetyl ethylhexanoate, cetyl isononanoate, cetearyl isononanoate, cetearyl nonanoate, ethylhexyl isononanoate, ethylhexyl isostearate and a mixture thereof.

6. The multilamellar vesicle containing retinal according to claim 1, wherein the average diameter of the multilamellar vesicle is 50 to 400 nm.

7. A cosmetic composition comprising the multilamellar vesicle containing retinal according to claim 1.

8. The cosmetic composition according to claim 7, which comprises 1 to 60% by weight of the multilamellar vesicle containing retinal.
